(19) United States
(12) Patent Application Publication
(10) Pub. No.: US 2005/0054099 A1
(43) Pub. Date: Mar. 10, 2005

(54) Matrix derived from whole organ

(75) Inventors: Yutaka Umehara, Hirosaki (JP); Jacek Rozga, Los Angeles, CA (US); Achilles A. Demetriou, Los Angeles, CA (US)

Correspondence Address: Richard H. Zaitlen, Esq.;Pillsbury Winthrop LLP, 725 South Figueroa Street, Suite 2800, Los Angeles, CA, 90017-5406, US

(73) Assignee: Cedars Sinai Medical Center, Los Angeles, CA (US)

(21) Appl. No.: 10/655,093

(22) Filed: Sep. 4, 2003

(51) Int. Cl. C12N 5/02

(52) U.S. Cl. 435/373; 435/325; 435/395

(57) ABSTRACT

Described herein is a novel biological support for cells, derived from a mammalian whole organ, tissue or portion thereof. In various embodiments, a support matrix is prepared by isolating a whole organ, tissue or portion thereof, and thereafter converting it into a tissue powder. Various washing and freeze-drying techniques are employed, as well as mechanical reduction, sonication and further processes to convert the whole organ, tissue or portion thereof into a matrix suitable for supporting functional longevity and proliferation of cultured cells. It is believed that by culturing a particular type of cell in a matrix derived from the whole organ, tissue or portion thereof with which that cell is normally associated, one may achieve optimal cell support and differentiation. The whole organs, tissues and portions thereof used in connection with aspects of the present invention need not be decellularized or otherwise digested prior to processing into the matrix.

### FIELD OF INVENTION

The present invention relates to the culturing of cells. More particularly, the invention includes a matrix to support the functional longevity and differentiation of cultured cells.

### BACKGROUND OF THE INVENTION

The culture of cells is a basic protocol that laboratory experiments and studies often require and employ on a daily basis. Maintaining cells in vitro provides a simple and safe way to test cell response in a variety of situations without requiring live subjects. Morphology and metabolic activity of cultured cells are affected by the composition of the substrate on which they are maintained and are believed to function best (e.g., perform their natural in vivo functions and/or proliferate) when cultured on substrates that closely mimic their natural environment. Currently, in vitro studies of cellular function are limited by the availability of cell growth substrates that effectively and affordably present an optimal physiologic environment for cultured cells.

The interaction of cells with their extracellular matrix in both in vivo and in vitro environments plays an important role in the function of cells from all organs. Continuous communication between cells and the surrounding matrix environment directs necessary processes such as the acquisition and maintenance of differentiated phenotypes during embryogenesis, the development of form, angiogenesis, wound healing, and even tumor metastasis. Both biochemical and biophysical signals from the extracellular matrix regulate fundamental cellular activities including adhesion, migration, proliferation, differential gene expression, and programmed cell death. Conversely, the cell can modify its extracellular matrix environment by modulating synthesis and degradation of specific matrix components.

The ability of complex substrates to support cell viability, functions and growth in vitro has been previously reported, and matrix products supporting cells in vitro are commercially available. Such complex substrates represent combinations of extracellular matrix components in a natural or processed form. For example, Human Extracellular Matrix and MATRIGEL Basement Membrane Matrix are both available from BD Biosciences Discovery Labware (Bedford, Mass.). Human Extracellular Matrix is a chromatographically partially purified matrix extract derived from human placenta and includes laminin, collagen IV and heparin sulfate proteoglycan. MATRIGEL is a soluble basement membrane extract of the Engelbreth-Holm-Swarm (EHS) tumor, gelled to form a reconstituted basement membrane. Both of these matrix products require costly biochemical isolation, purification, and synthesis techniques. Consequently, production costs are high.

Other, more recently developed matrices speak to improving the maintenance of cell cultures in vitro, but they are derived from decellularized tissue. This requires the costly steps of digestion or chemical breakdown of the organ used to produce the matrix. Therefore, there is a need in the art for a more comprehensive biological support, capable of enhancing cell proliferation and maintaining functional longevity, which is more affordable and easily produced than other currently available products.

### SUMMARY OF THE INVENTION

The invention pertains to an improved matrix and methods of making the matrix. The matrix reflects the natural interstitial environment of cells. It maintains cell viability and supports both functional longevity and differentiation. More specifically, the matrix is derived from a whole organ, a whole tissue or a portion thereof, and is believed to be a superior support matrix for cells that would normally constitute at least a portion of that organ or tissue in vivo. Moreover, the additional steps of enzymatic digestion or chemical breakdown of tissue may not be required in preparing a matrix in accordance with various embodiments of the present invention.

Furthermore, the matrix is illustratively demonstrated herein to achieve functional longevity and increased proliferation with hepatocytes. The liver has many functions, playing roles in detoxification, metabolism, and protein synthesis. However, regulation of hepatocyte DNA synthesis, cell growth and cell function are still unclear, making hepatocytes difficult cells to culture. That the matrix of the present invention can support hepatocyte viability, functional longevity and growth thus underscores its remarkable performance abilities.

An additional aspect of the invention relates to expanding the current knowledge and possibilities for artificial organs and organ parts. Current shortages with organ donors and problems with transplantations and grafts in injured organs create a great need for other therapeutic alternatives. The present invention may further research and enhance progress in this area, providing greater possibility that making and using artificial organs and organ parts will be likely in the future.

### BRIEF DESCRIPTION OF THE FIGURES

The file of this patent contains at least one drawing executed in color. Copies of this patent with color drawing(s) will be provided by the patent and Trademark Office upon request and payment of the necessary fee.

FIG. 1 is executed in color and illustrates the reduction of a harvested liver into a powder form, in accordance with an embodiment of the present invention. FIG. 1A depicts a harvested liver after it was sliced into 1-3 mm thickness, frozen in liquid nitrogen (N₂) and dried in a low pressure tank. FIG. 1B depicts the same freeze-dried liver in powder form, following grinding with a mortar and pestle.

FIG. 2 is executed in color and illustrates a morphological difference among hepatocytes cultured with a freeze-dried liver powder (“FDLP”) and those cultured on collagen, in accordance with an embodiment of the present invention. FIGS. 2A, 2C and 2E depict the morphology of hepatocytes cultured on a collagen-coated dish, at twenty-four hours, six days and fourteen days after plating, respectively. FIGS. 2B, 2D and 2F depict the morphology of hepatocytes cultured with FDLP, at twenty-four hours, six days and fourteen days after plating, respectively.

FIG. 3 illustrates albumin secretion at established time points after plating (Day 2, Day 4, Day 6, Day 10, Day 14) of hepatocytes cultured with FDLP and a control, in accordance with an embodiment of the present invention.

FIG. 4 illustrates urea synthesis at established time points after plating (Day 2, Day 4, Day 6, Day 10, Day 14) of hepatocytes cultured with FDLP and control collagen-coated dishes, in accordance with an embodiment of the present invention.

FIG. 5 is executed in color and illustrates the formation of a “hepatocyte sheet” four days after culturing cells with a matrix derived from a whole organ, in accordance with an embodiment of the present invention. FIG. 5A depicts the organization of hepatocytes into a three-dimensional structure and the formation of secondary structures in spite of weak cell attachment to the bottom of the culture plate. FIG. 5B depicts the ease with which hepatocytes of the present invention can be lifted off a culture plate as a sheet-like form with gentle pipetting after four days in culture.

FIG. 6 illustrates the small diameter of a microcarrier (around 3 μm) made of FDLP that is believed to be suitable to culture hepatocytes in order to yield a high density of cells, in accordance with an embodiment of the present invention. The measurement of the diameter is based on the following mathematical relationship: r=(⅔^{1/2}-1)R.

### DETAILED DESCRIPTION OF THE INVENTION

The invention is based on the inventors' discovery that a matrix derived from a whole organ, a whole tissue or a portion thereof demonstrates enhanced cell viability and differentiation, even in cell types that are difficult to culture. The discovery indicates that the commonly-used technique of decellularization in making commercially available matrices may not only be unnecessary, but perhaps even counterproductive.

In one embodiment of the present invention, cells are contacted, in vitro, with a matrix derived from an organ, under conditions conducive to cell growth and differentiation. The term “contacting,” as used herein with reference to cell culture, shall be understood as including direct and indirect contact between the cultured cells and the matrix, as in in vivo fluid communication. The term “conditions conducive to cell growth,” as used herein, refers to environmental conditions, such as sterile technique, temperature, nutrient supply or other conditions that will be readily apparent to those of skill in the art as being relevant to cell growth. Although the conditions used for culturing cells may depend on the particular cell type, cell growth conditions are generally well known in the art.

The invention provides a process by which to create a matrix (substratum) that supports viability and growth of isolated cells from various organs. “Supporting cell viability and growth,” as used herein, includes inducing cell viability and growth, maintaining cell viability and growth, cultivating cell viability and growth, or any other means of increasing or maintaining cell viability and count, as will be readily recognized by those of skill in the art. The described methods generate a product that achieves both functional longevity and greater cell differentiation.

The terms “isolated organ” and “isolated tissue” as used herein refer to an organ or tissue, respectively, which has been removed from a mammal. Any organ, tissue or portion thereof that is obtained from a mammal may be suitable for use in accordance with various embodiments of the present invention. By way of example, organs may include a liver, lung, kidney, pancreas, spleen, testis, intestinal wall, adrenal gland, thyroid gland, parathyroid gland, ovary or brain, while exemplary tissues might include skin, muscle, blood vessel wall or bone marrow. The aforementioned list of organs and tissues is by no means exhaustive; rather it is used to illustrate the broad array of organ and tissue types that may be used in connection with various embodiments of the present invention.

Correspondingly, a wide array of cell types may be cultured with a matrix of the present invention. It is believed that cells exhibit optimal growth and differentiation when cultured in a matrix derived from the same organ with which the cells being cultured are associated. Examples of cells that may be cultured with various matrices of the present invention may include, but are in no way limited to, hepatocytes, lung cells (e.g., lung alveolar cells), kidney cells (e.g., renal tubule cells), enterocytes, pancreatic islet cells (e.g., alpha, beta), splenocytes, neural cells and others.

The term “whole,” when used with reference to the organs and tissues included in various embodiments of the present invention, denotes the fact that these biological materials are not decellularized or otherwise digested prior to being processed into support matrices. Instead, such organs and tissues are processed in an undigested form. Moreover, use of the term “whole” is not intended to imply that a full and intact organ is necessarily processed; indeed, in many embodiments of the present invention only a portion of an organ is used to produce a matrix. By way of example, a segment of an intestine or a piece of skin may be utilized, just as a full, intact liver might be used. However, in each embodiment, the “whole” organ, tissue or portion thereof is processed in undigested form, whether the organ is a full and intact organ or tissue, or only a portion thereof.

Accordingly, the invention includes methods for reducing a whole isolated organ, tissue or portion thereof, through lyophilization, grinding and sonication (or other techniques), into a tissue powder. This powder constitutes the base component for the support matrix. The present invention takes advantage of a whole organ or tissue (or portion thereof) to provide a more comprehensive biological support for cell culture. While not wishing to be bound by any particular theory, it is believed that this ensures that many or all of the essential elements, which cells contact and interact with in vivo within specific organs, are present in the matrix. This results in a matrix that is able to support cell proliferation and maintain cell functional longevity to a greater extent than other methods currently available on the market. Furthermore, by using the whole organ or tissue (or portion thereof) to derive the tissue powder, the need to decellularize the organ by enzymatic digestion or chemical breakdown is obviated. Consequently, the invention offers an easier and more affordable way to obtain a matrix that is also more conducive to inducing cell proliferation and differentiation than other commercially available products.

### Isolation of Natural Organs of Interest and Process to Obtain Freeze-Dried Tissue Powder

The base component of the present invention is a tissue powder. This powder may be derived from a variety of organs or tissues. Depending on the cells to be cultured, an appropriate organ, tissue or portion thereof may be isolated after a preservation approach such as in situ perfusion using physiological saline, phosphate buffer solution, Hank's Balanced Salt Solution (“BSS”) or commercially available organ preservation media (e.g., University of Wisconsin solution); although other techniques may be employed to preserve cells for use with the matrix of the present invention, as will be readily understood by those of skill in the art.

After harvesting, blood may be removed from the organ, tissue or portion thereof, and the selected organ or tissue may be cleaned. The organ or tissue may be frozen in liquid nitrogen, and subsequently dried (e.g., in a low pressure tank). In alternate embodiments of the present invention, other conventional techniques may be used to freeze the selected organ or tissue. For example, the organ, tissue or portion thereof may be frozen using a helium-based technique. Additionally, the organ, tissue or portion thereof may be sliced or otherwise reduced into smaller pieces prior to freezing.

The resulting freeze-dried organ, tissue or portion thereof (whether reduced to smaller pieces or not) is next converted into powder. This may be achieved, for example, by grinding with a mortar and pestle. The powder may then be soaked in a culture medium and sonicated using a sonic dismembranator to further reduce the powder into finer particles. Other methods known in the art and comparable to those described may be used in the alternative. Due to the lypholization (freeze-drying) undergone by the powder in one embodiment of the present invention, it may be stored for future use without losing its potency.

### Preparing Support Matrix and Cell Culture

Cells to be cultured with the matrix of the present invention may be obtained by a variety of techniques, including in situ perfusion with collagenase, ethylenediaminetetraacetate (EDTA) or other solution causing cell separation. Enrichment of cells increases viability prior to suspension in a suitable medium, such as Dulbecco's Modified Eagle Medium (DMEM). An appropriate media may be selected based on a variety of factors, including, for example, the type of cell being cultured. Other examples of media that may be suitable for use with the cells and matrix of the present invention may include, but are in no way limited to, Williams E medium, Minimum essential medium (MEM) (Eagle), Ham's F10 medium, Ham's F12K medium and RPMI-1640 medium. Moreover, it will be readily apparent to those of skill in the art that any number of conventional cell growth media may be used in accordance with alternate embodiments of the present invention. Many cell growth media are commercially available and are used routinely to culture cells. Alternatively, one may easily create one's own cell growth medium and use the same in connection with various embodiments of the present invention.

Suspended cells are mixed with tissue powder that has been preferably soaked in the same culture medium as the cells, although soaking in an identical medium, indeed soaking in any medium at all, is not required for the tissue powder of the present invention to function properly. The mixture may be seeded on tissue culture plates and placed under conditions conducive to cell growth. About six hours after plating, various nutrients and/or antibiotics may be added to the culture medium. Subsequently, the medium may be periodically replaced (e.g., every 24 hours). The duration of time before additional components (e.g., nutrients, antibiotics, growth factors, etc.) are added, as well as the frequency with which the medium is replaced may vary with the type of cells being cultured, as well as other relevant factors that will be readily recognized by those of skill in the art.

### EXAMPLES

The liver was used as a model system to conduct proof-of-concept studies. The studies demonstrate that the instant invention provides a coating matrix as well as a micro-skeletal structure to support the cells. These are believed to be important features in culturing hepatocytes, which are anchorage-dependent and function through cell-to-cell contact. Primary studies show that even cell types that are considered difficult to culture (e.g., hepatocytes) successfully proliferate and maintain functional longevity with the methods of the present invention.

### EXAMPLE 1

Isolation of Liver and Preparation of Freeze-Dried Liver Powder (FDLP)

Adult male Sprague-Dawley rats (200-250g) were obtained from Harlan Sprague-Dawley Inc. (Indianapolis, Ind.). Animals were housed in a climate-controlled (21° C.) room under a 12-hours light-dark cycle and were given tap water and standard laboratory rat chow (Rodent Chow 5001, Ralston Purina; St. Louis, Mo.) ad libitum. Cell harvesting was performed between 9:00 a.m. and noon under general anesthesia (isoflurane) using sterile surgical technique. This study was performed in compliance with institutional and National Research Council guidelines for the care and use of experimental animals.

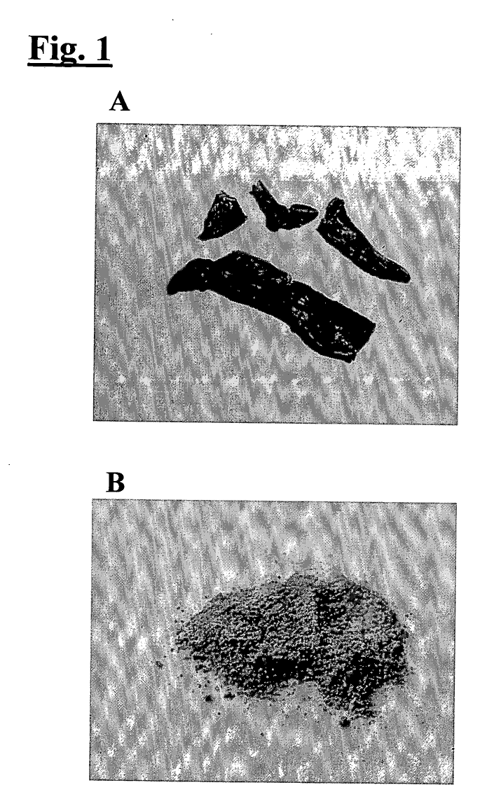

The rat liver was harvested after in situ perfusion with cold physiological saline (0.9% NaCl). The harvested liver was sliced into 1-3 mm thickness and frozen in liquid nitrogen. After freezing, the liver was dried in a low pressure tank (FIG. 1a). The freeze-dried liver was subsequently broken into powder using a mortar and pestle (FIG. 1b). Finally, the powder was soaked in a culture medium (DMEM) and sonicated using a sonic dismembranator (available from Fisher Scientific; Pittsburgh, Pa.) to further reduce particle size.

### EXAMPLE 2

### Hepatocyte Isolation

Hepatocytes were harvested by the Seglen in situ two-step liver perfusion method with some modifications (P.O. Seglen, “Preparation of Isolated Rat Liver Cells,” Methods Cell Biol. (Prescott, D. M., ed.) 13:29-83, Academic Press, New York (1976)). After enrichment through a PERCOLL density gradient (Pharmacia; Piscataway, N.J.), viability of the cells was always greater than 90%, as judged by trypan blue exclusion. The cells were next suspended in DMEM (Omega Scientific; Tarzana, Calif.) with 10% fetal bovine serum (FBS) (Sigma Chemical Co.; Saint Louis, Mo.).

### EXAMPLE 3

### Use of FDLP Matrix as Hepatocyte Cell Culture Substrate

Rat hepatocytes suspended in DMEM with 10% FBS were mixed with FDLP soaked in the same culture medium (1.5 mg/ml) and seeded on 60-mm non-coated tissue culture plates at densities of 4.5×10⁵ cells/ml and placed in a humidified, 5% CO₂; 95% air incubator at 37° C. Six hours after plating, the medium was replaced with DMEM enriched with 10% FBS, 20 mM HEPES, 10 mM nicotinamide, 1 mM ascorbic acid 2-phosphate, 10-7 M dexamethasone, 1 mg/ml galactose, 30 μg/ml proline, ITS mixture, 10 ng/ml epidermal growth factor (EGF) and antibiotics. The medium was replaced every 24 hours. In addition to the basal medium, 1% dimethyl sulfoxide (DMSO) was used from day four onward. Control dishes contained no FDLP and hepatocytes were cultured on 60-mm dishes coated with type I rat tail collagen (obtained from Collaborative Biomedical Products; Bedford, Mass.). Additionally, as a background, a culture plate containing only FDLP (1.5 mg/ml) was cultured with the same conditions. Five dishes from each culture group were used for a urea formation and albumin secretion study, and each cell culture experiment was repeated three times, using freshly isolated rat hepatocytes.

### EXAMPLE 4

### Measurements of Cell Growth through Urea Formation and Albumin Secretion

The culture medium was collected at 2, 4, 6, 10, and 14 days after plating and analyzed for albumin concentration using an ELISA kit (Nephrat II, Exocell Inc.; Philadelphia, Pa.). The antibodies in this kit were not cross-reactive with bovine albumin. The amount of albumin secreted into the culture medium during the last 24 hours was calculated.

20 mM ammonium chloride was spiked at 2, 4, 6, 10 and 14 days after plating. After 6 hours of incubation, samples were collected and ammonia and urea concentrations were measured, using an enzymatic test kit (Boehringer Mannheim GmbH; Biochemica, Germany) with 340 nm spectrometer (BECKMAN model DU 530, Beckman Instruments, Inc.; Fullerton, Calif.).

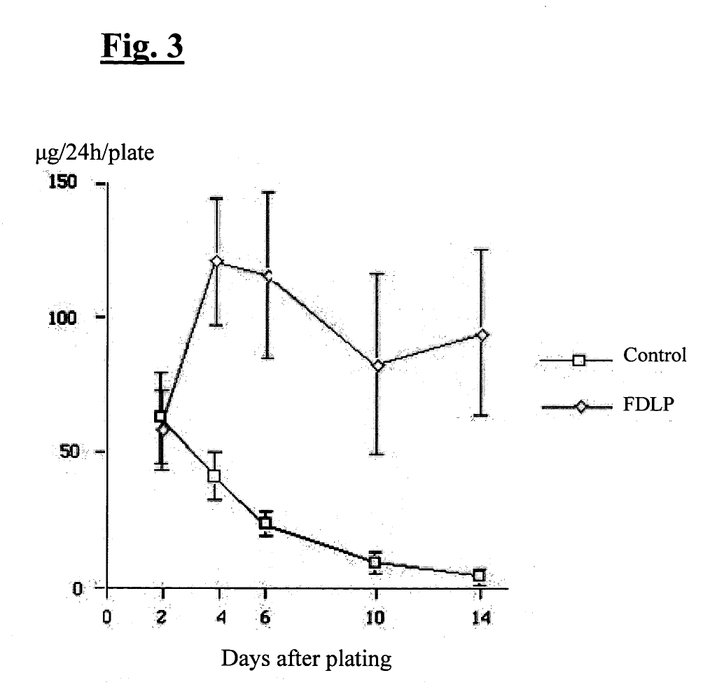

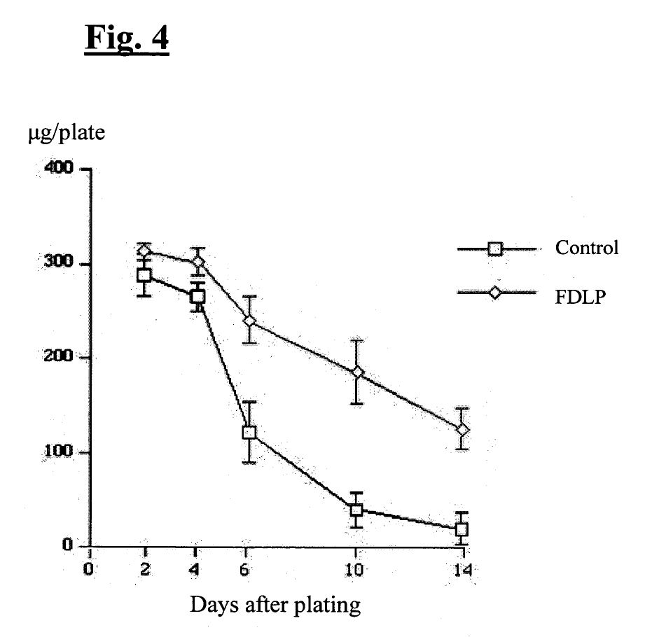

Data were analyzed using Student's t test (P values equal to or less than 0.05 were considered significant). Resulting values are presented as means±SD (FIGS. 3-4).

### EXAMPLE 5

### Comparison of Urea Formation and Albumin Secretion

At 2 days after plating, there was no significant difference between hepatocytes cultured with FDLP and the control (FDLP group, 57.9±14.7 μg/24 h/plate; control group, 62.7±17.0 μg/24 h/plate). Hepatocytes cultured with FDLP secreted maximum amounts of albumin at 4 days after plating and kept the level until 14 days after plating (Day 4, 120.8±23.3 μg/24 h/plate; Day 6, 115.8±30.6 μg/24 h/plate; Day 10, 82.8±33.6 μg/24 h/plate; Day 14, 94.2±31.1 μg/24 h/plate). In the control culture, the amount of secreted albumin decreased rapidly and reached almost zero at 14 days after plating (Day 4, 41/1±8.5 μg/24 h/plate; Day 6, 23.7±4.8 μg/24 h/plate; Day 10, 9.7±3.7 μg/24 h/plate; Day 14, 3.9±3.2 μg/24 h/plate) (FIG. 3). The background, culturing FDLP with no cells, was zero at every time point.

When the hepatocytes with FDLP and the control culture were given 2.0 mM of ammonium chloride, the amount of urea synthesized in 6 hours decreased according to the age in both cultures. However, the decrease rate was much slower in the FDLP group (FDLP group, Day 2, 312.3±9.5 μg/6 hours; Day 4, 300.7±13.4 μg/6 hours; Day 6, 240.0±25.9 μg/6 hours; Day 10, 184.4±38.9 μg/6 hours; Day 14, 123.3±22.3 μg/6 hours; control group, Day 2, 287.3±22.6 μg/6 hours; Day 4, 264.9±15.0 μg/6 hours; Day 6, 121.0±31.3 μg/6 hours; Day 10, 39.0±17.8 μg/6 hours; Day 14, 17.8±14.6 μg/6 hours) (FIG. 4). 14 days after plating, the hepatocytes cultured with FDLP maintained the same level of metabolic function as the hepatocytes on collagen, 6 days after plating. The background, culturing FDLP with no cells, was zero at every time point.

### EXAMPLE 6

### Morphological Appearance of Cells

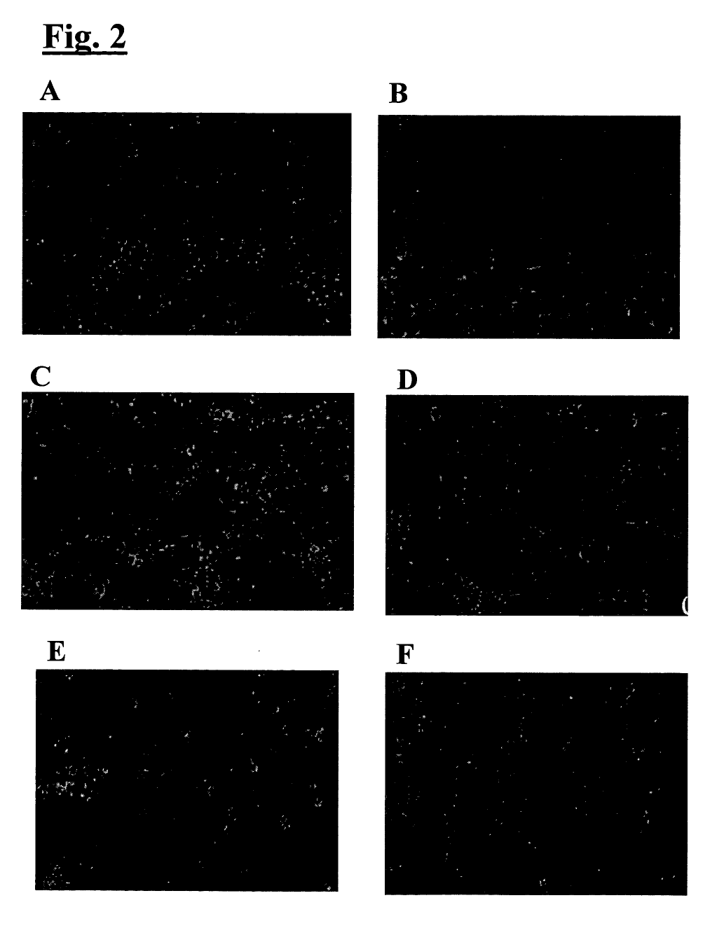

Morphology of the hepatocytes cultured with FDLP was significantly different from that of the hepatocytes cultured on collagen. At 24 hours after plating, in contrast to hepatocytes cultured on collagen, those cultured with FDLP showed minimal spreading and formed aggregates (FIG. 2b). At Day 6, the hepatocytes attached to the bottom of the dish and made islet-like structures. The cells tended to pile up into three-dimensional spheroids (FIG. 2d). These spheroids were maintained until 14 days after the plating (FIG. 2f).

### EXAMPLE 7

### Tissue-Like Cell Formation

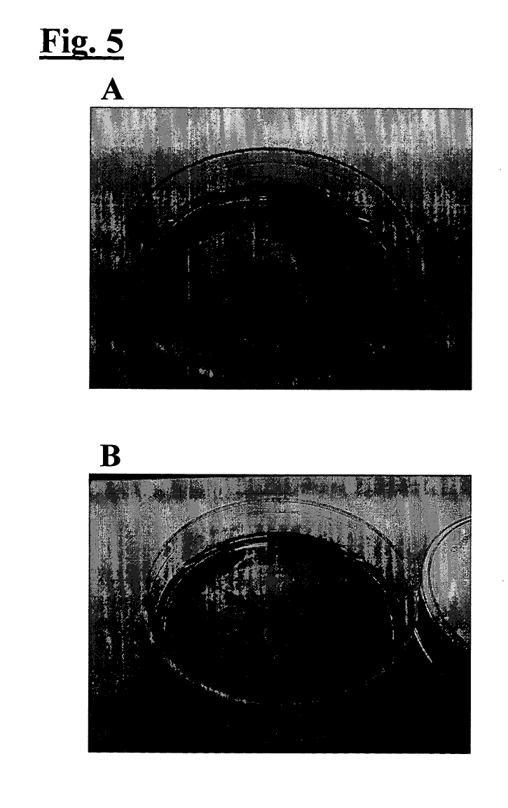

Results from studies show greater growth in cells cultured in the instant matrix than in those cultured in the traditional matrix or the control. More promising, the cells cultured through the invention organized into three-dimensional structure with tight junctions (within 6-7 days) and formed secondary structures such as ducts and bile canaliculi. The result was the formation of a “hepatocyte sheet” after only 4 days of plating (FIG. 5).

### EXAMPLE 8

### Applications in Bioengineering

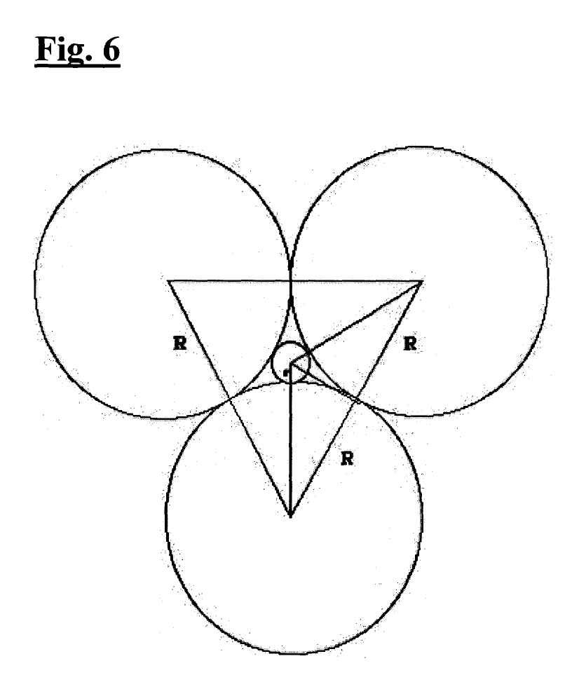

Microcarrier-attached hepatocytes are unique culture systems, and the ideal diameter of a microcarrier needed to achieve the highest density of hepatocytes is very small (approx. 3 μm) (FIG. 6). It is difficult to make such a small artificial particle which contains a matrix, even with the technology we have available today. In this regard, FDLP is thought to have significant potential advantages in its size and use as a matrix.

One interesting aspect of this culture system, using FDLP as a microcarrier, is that hepatocytes can make spheroids despite the fact that cell attachment to the bottom of the culture plate is weak. The inventors used non-coated plastic culture plate for hepatocyte culture using FDLP. Four days after plating, hepatocytes may be readily lifted off from the plate as a sheet-like form with gentle pipetting (FIG. 5). This “hepatocyte sheet” is thought to have many benefits that may allow wide use in research and in different applications in the field of tissue engineering. For example, cells cultured in this fashion may be used to fabricate artificial organs or components thereof for transplantation.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

1. A cell culture matrix composition, comprising:
a tissue powder derived from a whole organ, a whole tissue, a portion of a whole organ or a portion of a whole tissue.

2. The composition of claim 1, wherein
the organ is a liver, a lung, a kidney, a pancreas, a spleen, a testis, an intestinal wall, an adrenal gland, a thyroid gland, a parathyroid gland, an ovary or a brain, and
the tissue is skin, muscle, a blood vessel wall or bone marrow.

3. The composition of claim 1, further comprising a medium.

4. A method for producing a tissue powder, comprising:
isolating a biological material selected from the group consisting of a whole organ, a whole tissue, a portion of a whole organ and a portion of a whole tissue; and
converting the biological material into the tissue powder.

5. The method of claim 4, wherein converting the biological material further includes grinding with a mortar and pestle to produce ground particles.

6. The method of claim 5, wherein converting the biological material further includes sonicating the ground particles to produce the tissue powder.

7. The method of claim 6, wherein sonicating the ground particles is performed with a sonic dismembranator.

8. The method of claim 6, wherein, prior to sonicating the ground particles, the method further includes soaking the tissue powder in a medium.

9. The method of claim 4, wherein the biological material is a liver, a lung, a kidney, a pancreas, a spleen, a testis, an intestinal wall, an adrenal gland, a thyroid gland, a parathyroid gland, an ovary, a brain, skin, muscle, a blood vessel wall or bone marrow.

10. The method of claim 4, wherein isolating a biological material further includes employing a perfusion technique.

11. The method of claim 4, wherein after isolating the biological material, the method further includes freezing the biological material.

12. The method of claim 4, wherein, after isolating the biological material, the method further includes drying the biological material in a low-pressure tank.

13. The method of claim 11, wherein, after isolating the biological material but prior to freezing the biological material, the method further includes reducing the biological material into smaller pieces.

14. A method of supporting cell growth, comprising:
providing cells; and
suspending the cells in a medium with a tissue powder derived from a biological material selected from the group consisting of a whole organ, a whole tissue, a portion of a whole organ and a portion of a whole tissue.

15. The method of claim 14, wherein providing cells further includes harvesting the cells from a mammal.

16. The method of claim 14, wherein the cells are of the same type as those which at least partially constitute the biological material.

17. The method of claim 14, wherein providing the cells further includes employing a perfusion technique.

18. The method of claim 14, wherein the method further includes periodically replacing the medium.

19. The method of claim 14, wherein the biological material is a liver, a lung, a kidney, a pancreas, a spleen, a testis, an intestinal wall, an adrenal gland, a thyroid gland, a parathyroid gland, an ovary, a brain, skin, muscle, a blood vessel wall or bone marrow.

20. The method of claim 14, wherein the cells are hepatocytes, lung cells, kidney cells, enterocytes, pancreatic islet cells, splenocytes, or neural cells.

21. The method of claim 14, wherein the cell growth is supported to provide cells for creating an organ to use in an organ replacement or transplantation.
